(19) United States
(12) Patent Application Publication
(10) Pub. No.: US 2021/0313011 A1
(43) Pub. Date: Oct. 7, 2021

(54) GENOMIC SEQUENCING SELECTION SYSTEM

(71) Applicant: Quest Diagnostics Investments LLC, Secaucus, NJ (US)

(72) Inventors: Anindya Bhattacharya, San Juan Capistrano, CA (US); Anna Gerasimova, San Juan Capistrano, CA (US); Quoclinh Nguyen, San Juan Capistrano, CA (US); Christopher Elzinga, Marlborough, MA (US); Edward Moler, San Juan Capistrano, CA (US)

(73) Assignee: Quest Diagnostics Investments LLC, Secaucus, NJ (US)

(21) Appl. No.: 17/286,310

(22) PCT Filed: Oct. 16, 2019
(86) PCT No.: PCT/US2019/056479

§ 371 Date: Apr. 16, 2021

Related U.S. Application Data
(60) Provisional application No. 62/766,432, filed on Oct. 17, 2018.

(51) Int. Cl. G16B 30/00; G16B 50/00; G16B 20/20

(52) U.S. Cl. CPC G16B 30/00; G16B 20/20; G16B 50/00

(57) ABSTRACT

The systems and methods discussed herein can calculate sequencing statistics such as coverage depth for sequencing data. The present solution can determine variant frequencies and identify clinically relevant variants. The present solution can read BAM and VCF input files and Phred scaled quality scores. The present solution can select relatively high quality reads based on the quality scores and can calculate reference and alternative allele counts for SNPs, insertions and deletions (INDELs), and structural variants.

### CROSS-REFERENCE TO RELATED APPLICATION

This application claims priority to and the benefit of U.S. Provisional Patent Application No. 62/766,432, titled “GENOMIC SEQUENCING SELECTION SYSTEM,” and filed Oct. 17, 2018, the content of which is hereby incorporated herein by reference in its entirety for all purposes.

### BACKGROUND OF THE DISCLOSURE

Genomic sequencing systems, including next-generation sequencing (NGS) systems (sometimes referred to as massively parallel sequencing systems or by similar terms), can produce large quantities of sequencing data of variable quality. Specifically, in many implementations, an NGS system can fragment a genome into a plurality of small segments. These small segments can be sequenced in parallel, reducing processing requirements relative to sequencing the entire genome as a whole, and then may be recombined to generate a complete sequence. Sequence metrics can be calculated on the sequencing data.

NGS systems provide much faster and less expensive sequencing compared to first-generation sequencing techniques such as Sanger sequencing. However, NGS systems suffer from inaccuracies or noise due to errors in identification of base sequences or base calling, or errors introduced during sample preparation. Error rates in base reads may be 10% or more, sometimes as high as 25% or more. Given the immense amount of data that may be obtained in a short time by an NGS system, even moderate error rates may result in data with hundreds of thousands or even millions of incorrect base pairs.

### SUMMARY OF THE DISCLOSURE

The systems and methods disclosed herein provide for measurement of error rates and read quality on a read-by-read basis, and in some implementations may filter or exclude low quality reads or extract high quality reads and provide detailed metrics. This may reduce processing requirements compared to analyzing entire data sets including low quality or erroneous data and can increase computational speeds of determining sequence metrics by reducing the amount of computational time spent on data that may provide inaccurate results. In many implementations, these systems and methods may also reduce memory and bandwidth consumption relative to processing or transferring data sets with high error rates.

In some implementations, the present solution can calculate sequencing statistics such as coverage depth. The present solution can determine read statistics such as variant frequencies and identify clinically relevant variants. The present solution can read BAM and VCF input files and Phred scaled quality scores. The present solution can select relatively high quality reads based on the quality scores and can calculate reference and alternative allele counts for single nucleotide polymorphisms (SNPs), insertions and deletions (INDELs), and structural variants. The present solution can calculate the sequencing metrics for different strands to measure strand bias. The present solution can also determine minimum, maximum, and mean depths for each region of the sequence data.

According to at least one aspect of the disclosure, a method to filter sequencing data can include receiving, by a data processing system, data that can include a plurality of gene sequences. Each of the plurality of gene sequences can include an indication of a chromosome, an indication of a position, a base value, and a quality score. The method can include selecting, by the data processing system, a subset of the plurality of gene sequences. Each of the subset of the plurality of gene sequences can have the same indication of the chromosome. The method can include filtering, by the data processing system, from the subset of the plurality of gene sequences, gene sequences comprising base values that have the quality score above a predetermined threshold. The method can include determining, by the data processing system, an aggregate count for each position of the filtered gene sequences. The method can include determining, by the data processing system, an alternative base count for each position of the filtered gene sequences. The method can include generating, by the data processing system, an identification of a gene sequence variant based on a ratio of the alternative base count for each position to the aggregate count for each position exceeding a threshold.

In some implementations, the method can include determining an alternate count for a deletion sequence in the filtered subset of the plurality of gene sequences where the base values have the quality score above the predetermined threshold. The deletion sequence can start at an index neighboring the position.

The method can include determining an alternate count for an insertion sequence in the filtered subset of the plurality of gene sequences where the base values have the quality score above the predetermined threshold. The method can include determining the alternate count for the insertion sequence further by identifying an alternate sequence match. The method can include identifying a structural variant in the filtered plurality of gene sequences.

In some implementations, the alternative base count can be determined based on the structural variant identified in the plurality of gene sequences. Determining the aggregate count can include counting a match in each of the filtered subset of the plurality of gene sequences with a CIGAR string.

In some implementations, determining the aggregate count can include counting a deletion, insertion, reference skip, soft clip, or hard clip in each of the filtered subset of the plurality of gene sequences. The method can include calculating at least one of a mean read coverage, a max read coverage, or a maximum read coverage for the filtered plurality of gene sequences based on the aggregate count and the alternative base count.

In some implementations, the method can include calculating a strand bias for the plurality of gene sequences based on the aggregate count and the alternative base count.

According to at least one aspect of the disclosure, a system to filter sequencing data can include a data processing system. The system can receive data that can include a plurality of gene sequences. Each of the plurality of gene sequences can include an indication of a chromosome, an indication of a position, a base value, and a quality score. The system can select a subset of the plurality of gene sequences. Each of the subset of the plurality of gene sequences can have the same indication of the chromosome. The system can filter, from the subset of the plurality of gene sequences, gene sequences in which the base values have the quality score above a predetermined threshold. The system can determine an aggregate count for each position of the filtered subset of the plurality of gene sequences where the base values have the quality score above the predetermined threshold. The system can determine an alternative base count for each position of the filtered plurality of gene sequences where the base values have the quality score above the predetermined threshold. The system can identify gene sequence variants based on a ratio of the alternative base count for each position to the aggregate count for each position, and may generate an identifier of the gene sequence variants.

In some implementations, the system can determine an alternate count for a deletion sequence in the subset of the plurality of gene sequences where the base values have the quality score above the predetermined threshold. The system can determine an alternate count for an insertion sequence in the filtered subset of the plurality of gene sequences where the base values have the quality score above the predetermined threshold.

In some implementations, the system can determine the alternate count for the insertion sequence by identifying an alternate sequence match. The system can identify a structural variant in the plurality of gene sequences.

The system can determine the aggregate count by counting a match in each of the filtered subset of the plurality of gene sequences with a CIGAR string. The system can determine the aggregate count by counting a deletion, insertion, reference skip, soft clip, or hard clip in each of the subset of the plurality of gene sequences.

The system can calculate at least one of a mean read coverage, a max read coverage, or a maximum read coverage for the plurality of gene sequences based on the aggregate count and the alternative base count. The system can calculate a strand bias for the plurality of gene sequences based on the aggregate count and the alternative base count.

The foregoing general description and following description of the drawings and detailed description are exemplary and explanatory and are intended to provide further explanation of the invention as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates a block diagram of an example system to compute NGS read depth statistics.

FIG. 2 illustrates a block diagram of an example method to determine coverage metrics of sequencing data using the system illustrated in FIG. 1.

FIG. 3 illustrates example sequence listings for a given chromosome.

FIG. 4 illustrates a block diagram of an example computer system.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present solution can calculate sequencing statistics such as coverage depth. The present solution can determine variant frequencies and identify clinically relevant variants based on the variant frequencies. The present solution can read BAM and VCF input files and Phred scaled quality scores. The present solution can select relatively high quality reads from the input files based on the quality scores and can calculate reference and alternative allele counts for SNPs, insertions and deletions (INDELs), and structural variants. The present solution can calculate the sequencing metrics for different strands to measure strand bias. The present solution can also determine minimum, maximum, and mean depths for each region of the sequence data. The present solution can use the quality scores to select and analyze only relatively high quality reads, which can increase computational speeds of determining sequence metrics by reducing the amount of computational time spent on data that may provide inaccurate results.

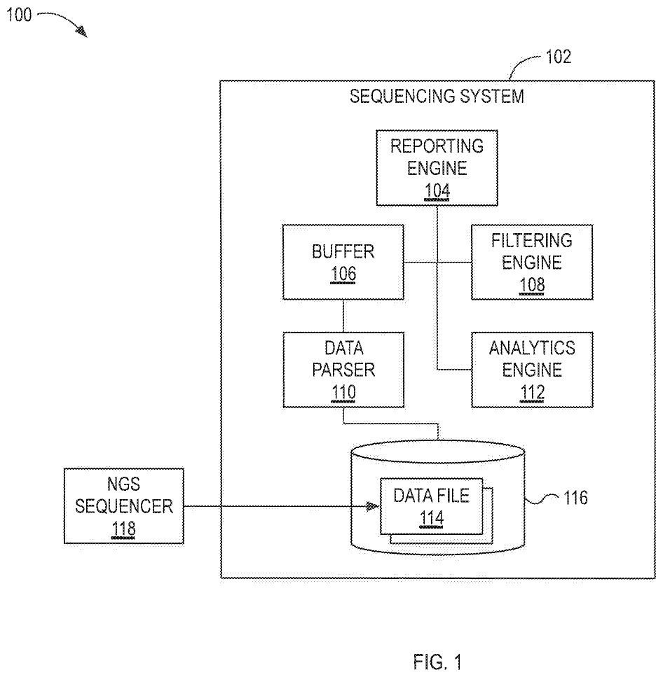

FIG. 1 illustrates a block diagram of an example system 100 to compute NGS read depth statistics. The system 100 can include a sequencing system 102. The sequencing system 102 can include a data parser 110 that reads data files 114 from a data repository 116. The data parser 110 can load the data into a buffer 106. The sequencing system 102 can include a reporting engine 104, a filtering engine 108, and an analytics engine 112. The system 100 can include an NGS sequencer 118 that can provide the data files 114 to the sequencing system 102.

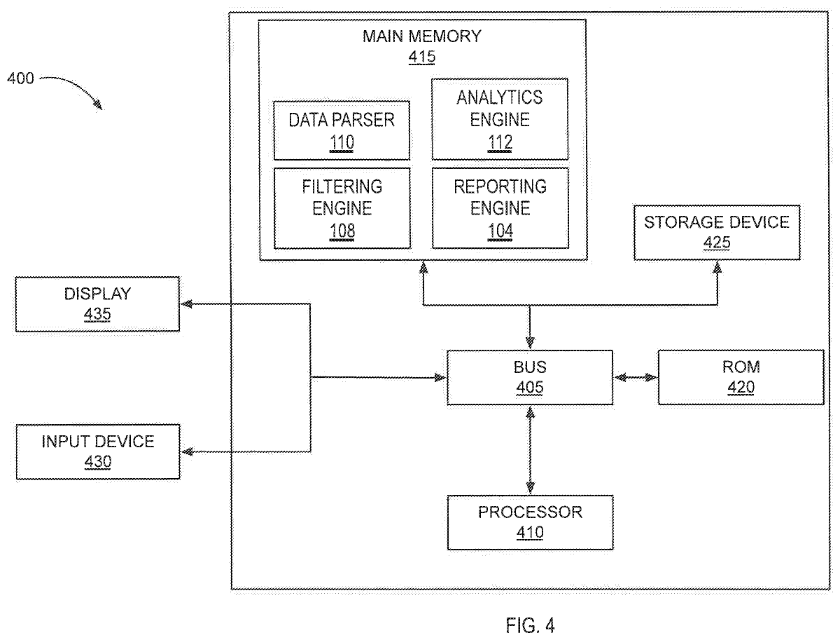

The system 100 can include a sequencing system 102. The sequencing system 102 can include at least one server or computer having at least one processor. For example, the sequencing system 102 can include a plurality of servers located in at least one data center or server farm or the sequencing system 102 can be a desktop computer. The processor can include a microprocessor, application-specific integrated circuit (ASIC), field-programmable gate array (FPGA), other special purpose logic circuits, or combinations thereof. The sequencing system 102 can be a data processing system as described in relation to FIG. 4. For example, the sequencing system 102 can include one or more processors and memory. The sequencing system 102 can include a user interface (e.g., a graphical user interface) that is rendered and displayed to the user via a display coupled with the sequencing system 102. One or more input/output (I/O) devices can be coupled with the sequencing system 102.

The sequencing system 102 can include the data repository 116. The data repository 116 can include one or more local or distributed databases. The data repository 116 can include computer data storage or memory and can store one or more data files 114. The data repository 116 can include non-volatile memory such as one or more hard disk drives (HDDs) or other magnetic or optical storage media, one or more solid state drives (SSDs) such as a flash drive or other solid state storage media, one or more hybrid magnetic and solid state drives, one or more virtual storage volumes such as a cloud storage, or a combination thereof.

The sequencing system 102 can store one or more data files 114 in the data repository 116. Each of the data files 114 can include a plurality of gene sequence data. The gene sequence data can include an indication of a chromosome, an indication of a position, a base value, and a quality score.

The data files 114 can be data files that are in the variant call format (VCF), sequence alignment mapping (SAM) format, binary sequence alignment mapping (BAM), of other file data file formats used in bioinformatics. For example, the data files 114 can include text data or binary data. In some implementations, the data files 114 can include strings of sequencing data. In some implementations, the data files 114 can include sequencing data that identifies the differences between a reference sequence and a sample sequence.

For example, the VCF file format can be used to store sequence variations. The VCF file format can be used to store single nucleotide polymorphisms (SNP), short (e.g., less than 10 base pairs) insertions and deletions, and large structural variants. The VCF file format (and other file formats) can include a header section and a body section. The header section can include metadata that further describes the data within the body of the VCF file format. The body of the VCF file format can include a plurality of columns. Each row can indicate a variation. The columns can identify the chromosome on which the variation is called; a position of the variation in the sequence; an identifier of the variation; a reference base value for the position; an alternative base value for position (e.g., which base other than the reference base was read at the position); a score; and a flag indicating which of a given set of filters the variation passed.

The sequencing system 102 can include an NGS sequencer 118. The NGS sequencer 118 can generate the data files 114. The system 100 can include a plurality of NGS sequencers 118. The NGS sequencer 118 can be provided samples from which the NGS sequencer 118 generates sequencing data. The NGS sequencer 118 can save the data into one of the above-described file formats. In some implementations, the NGS sequencer 118 can transmit the data files 114 to the sequencing system 102 via a network. In some implementations, the NGS sequencer 118 can transmit the data files 114 to an intermediary device such as cloud-based storage or a removable hard drive. The data files 114 can be transferred from the intermediary device to the sequencing system 102.

The sequencing system 102 can include a data parser 110. The data parser 110 can be any script, file, program, application, set of instructions, or computer-executable code that is configured to enable a computing device on which the data parser 110 is executed to read and extract data from the data repository 116. The data parser 110 can read the data files 114 from the data repository 116. In some implementations, the data files 114 can be stored in the data repository 116 in a compressed format. The data parser 110 can decompress the data files 114 before extracting the sequencing data from the data files 114. The data parser 110 can read the data files 114 from the data repository 116, which can be stored on the hard drive of the sequencing system 102. The data parser 110 can load the data files 114 and store the data from the data files 114 in the buffer 106.

In some implementations, the data parser 110 can load one or more data files 114 into the buffer 106. The data parser 110 can parse or process the data before the data parser 110 loads the data into the buffer 106. For example, the data parser 110 can parse the body of the VCF file format into one or more dictionaries or other file structure formats.

The sequencing system 102 can include a buffer 106. The buffer can be stored in random access memory (RAM) or other cached memory. The buffer can be stored on volatile memory. In some implementations, reading and writing to the buffer 106 can be faster than reading or writing to the data repository 116. The data parser 110 can load the data files 114 into the buffer 106 to reduce the number of reads and writes that are performed on the data repository 116 to improve the overall calculation speeds of the sequencing system 102.

The sequencing system 102 can include a filtering engine 108. The filtering engine 108 can be any script, file, program, application, set of instructions, or computer-executable code that is configured to enable a computing device on which the filtering engine 108 is executed to select variants from the sequencing data loaded into the buffer 106. As described above, each variation can include a score. The score can be a quality score. The quality score can be a Phred quality score. The quality score can be an indication of the quality of the base identified during the sequencing process. For example, the quality score can be an indication of the likelihood that the base at the given position was correctly identified and was not a sequencing error.

The filtering engine 108 can select only the variations that have a quality score above a predetermined threshold. For example, the filtering engine 108 can discard from the buffer 106 or from further analysis the variations with a quality score below the predetermined threshold. In some implementations, the filtering engine 108 does not use any variations with a Phred quality score less than 60, less than 50, less than 40, less than 30, or less than 20. In some implementations, the quality score can be based on the average reads per base in the sequencing data. For example, the quality score threshold can initially be set to 30 and then can be lowered if the average reads per base is above 100.

The sequencing system 102 can include an analytics engine 112. The analytics engine 112 can be any script, file, program, application, set of instructions, or computer-executable code that is configured to enable a computing device on which the analytics engine 112 is executed to calculate sequencing statistics.

The analytics engine 112 can calculate alternative base frequencies at each of the positions (P) indicated in the data files 114. The alternative base frequencies can be based on a count of all the reads at a given position. For example, the analytics engine 112 can determine the number of times each base occurs at each position in the gene sequence (or portion thereof), which can be referred to as an ALT base count for the given base. The analytics engine 112 can determine an aggregate count for each position in the gene sequence (or portion thereof). In some implementations, the analytics engine 112, when determining the ALT base count and the aggregate base count, may only include or count bases with a quality score above a predetermined threshold.

The analytics engine 112 can calculate alternative base frequencies for insertions and deletions. In some implementations, the insertions or deletions are less than 10 base pairs long. For deletions, the analytics engine 112 can determine the ALT count by identifying each of the deletions of a given length K that start at the position P+1. For insertions, the analytics engine 112 can determine the ALT count by counting the number of occurrences of an insertion of a given length that match a CIGAR string. For large structural variants, the analytics engine 112 can determine a reference (REF) count, an ALT count, and an aggregate or total count. The analytics engine 112 can determine the REF count as the number of occurrences that analytics engine 112 identifies that match to a CIGAR string across an event boundary. The analytics engine 112 can determine the ALT count as the number of deletions, insertions, reference skips, soft clips, or hard clips in the CIGAR across the event boundary. The total count can be the sum of the REF count and the ALT count. Based on the statistics and other data determined by the analytics engine 112, the analytics engine 112 can identify clinically relevant variants from common variants.

The sequencing system 102 can include a reporting engine 104. The reporting engine 104 can be any script, file, program, application, set of instructions, or computer-executable code that is configured to enable a computing device on which the reporting engine 104 is executed to generate reports based on the data generated by the analytics engine 112. The reporting engine 104 can receive the data generated by the analytics engine 112, such as the ALT count, REF count, and ALT frequencies. The reporting engine 104 can generate reports based on the data. The reporting engine 104 can determine and include in the report's coverage frequencies; strand bias; and mean, max, and average coverage.

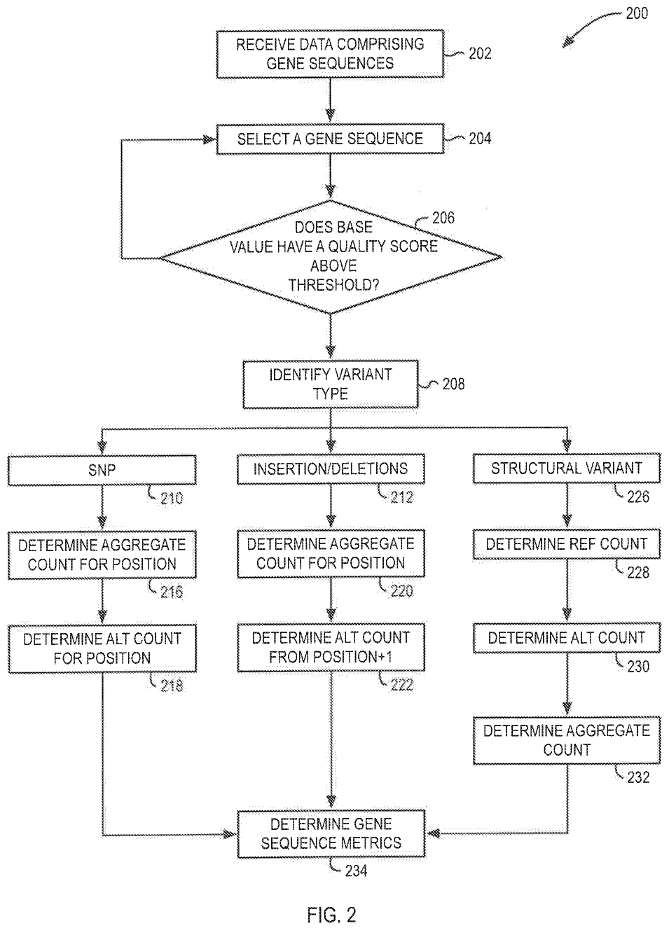

FIG. 2 illustrates a block diagram of an example method 200 to determine coverage metrics of sequencing data. The method 200 can include receiving data (BLOCK 202). Also referring to FIG. 1, the sequencing system 102 can receive the data. The sequencing system 102 can receive the data from the NGS sequencer 118 or the sequencing system 102 can retrieve the data from the data repository 116. The sequencing system 102 can receive the data as BAM, VCF, txt, or other file format that can contain sequencing data. The sequencing system 102 can also receive Phred scaled quality scores for the received data. The data can include a plurality of gene sequences. The data can indicate a chromosome for the gene sequence, position data, base values at each of the positions, and quality scores for the base values. In some implementations, the sequencing system 102 can receive and open the data files. The sequencing system 102 can read the data files into the buffer 106. Reading the data files into the buffer 106 can reduce the number of reads that are made to the data repository 116.

The method 200 can include selecting a gene sequence (BLOCK 204). The sequencing system 102 can select one or more gene sequences that belong to the same chromosome. In some implementations, the sequencing system 102 can select one or more gene sequences that also belong to the same general location on the chromosome or same specific location. For example, the gene sequences can be received in data files that include a plurality of columns. One of the plurality of columns can indicate a chromosome for the sequence data contained in another column of the data file. The sequencing system 102 can filter through the data to select the gene sequences that below to a predetermined chromosome.

The method 200 can include determining whether each base value has a threshold above a threshold (BLOCK 206). The sequencing system 102 can identify base values in the sequence data that include base values at a given position that are below the quality threshold. The sequencing system 102 can discard loaded data for the given position where the base value has a quality score below the predetermined threshold. The sequencing system 102 can save the base values for a given position that have a quality score above the predetermined threshold to a data structure, such as a dictionary that is saved to the buffer 106.

The method 200 can include identifying a variant type in the sequence data (BLOCK 208). The sequencing system 102 can determine whether the variant is a single nucleotide polymorphism (SNP) and continue to BLOCK 210, an insertion or deletion and continue to BLOCK 212, or a large structural variant and continue to BLOCK 226. In some implementations, the insertions or deletions are less than 10 base pairs (bp), and the large structural variants are greater than 10 base pairs.

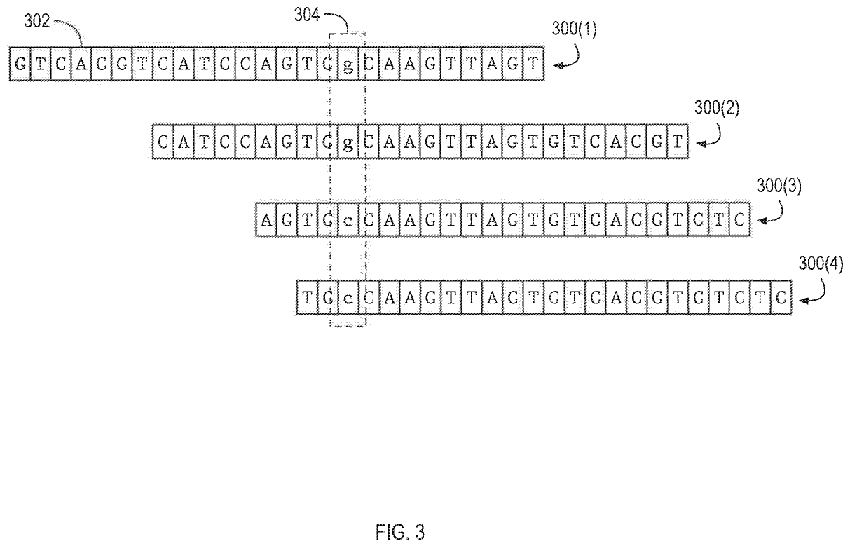

If the sequencing system 102 determines that the variant is a SNP, the method 200 can include determining an aggregate count for the position (BLOCK 216). Also referring to FIG. 3, among others, FIG. 3 illustrates four sequence listings 300(1)-300(4) (that are generally referred to as sequence listings 300) for a given chromosome. Each of the sequence listings 300 can include a plurality of base pairs 302. Each of the selected sequence listings 300 can overlap a given base pair position 304. Generically, the location of a base pair 302 can be described with the variable P where the next base pair 302 has the location P+1 and the previous base pair 302 has the location P−1. In this example, the data files can indicate the SNP occurs at the base pair position 304, which can be referred to as P. For example, sequence listing 300(1) and sequence listing 300(2) indicate that the base pair at base pair position 304 should be G and the sequence listing 300(3) and the sequence listing 300(4) indicate that the base pair at base pair position 304 should be C. Each of the base pairs 302 at the base pair position 304 can have an associated quality score.

The aggregate count for a position P can be the number of sequence listings 300 that include the position P with a quality score above the predetermined threshold. For example, and continuing the above example illustrated in FIG. 3, if the base pair 302 in the sequence listing 300(4) at the base pair position 304 have a quality score below the predetermined threshold, the aggregate count for the base pair position 304 can be 3.

The method 200 can include determining the alternative (ALT) count for the position (BLOCK 218). The sequencing system 102 can determine an ALT count for each base pair (e.g., C, G, G, and T). The ALT count for each base pair location 304 can be the aggregate count or the number of occurrences of the base pair at the base pair location 304. The sequencing system 102 may only include base pairs 302 in the ALT count that have a quality score above the predetermined threshold. For example, and referring to the example illustrated in FIG. 3, the sequencing system 102 can determine the ALT count for G at the base pair location 304 is 2 and the ALT count for C at the base pair location 304 is 1. The ALT count for C at the base pair location 304 is not 2 because as discussed above, in this example, the base pair 302 at the base pair location 304 in the sequence listing 300(4) has a quality score below the predetermined quality score threshold and is not considered in the calculations made by the sequencing system 102.

If, at BLOCK 208, the sequencing system 102 determines the variant type is an insertion or deletion, the method 200 can continue to BLOCK 212. The method 200 can include determining an aggregate count for each position (BLOCK 220). As described in relation to BLOCK 216 and BLOCK 218, the sequencing system 102 can count only the base pairs with a quality score above the predetermined threshold when determining the aggregate count for each position.

The method 200 can include determining the ALT count (BLOCK 222). For a deletion, the ALT count can be determined for the location of P+1. For example, the ALT count can be the number of deletions with a deletion length of K at the CIGAR position P+1. For an insertion, the ALT count can be the count of the number of reads with length L at CIGAR starting position P+1 and an alternative sequence match that matches the base pair read at P+1.

If, at BLOCK 208, the sequencing system 102 determines the variant type is a structural variant the method 200 can continue to BLOCK 226. The method 200 can then include determining a reference (REF) count (BLOCK 228). When determining the REF count, the sequencing system 102 can only count base pair reads with a quality score above the predetermined threshold. The structural variant can span an event boundary that starts at an event start in the gene sequence and ends at an event end in the gene sequence. The sequencing system 102 can determine the REF count as the number of reads that match in the CIGAR over the event boundary.

The method 200 can include determining an ALT count (BLOCK 230). When the variant type is a structural variant, the sequencing system 102 can determine the ALT count as the occurrences of deletions, insertions, reference skips, soft clips, or hard clips in the CIGAR across the event boundary.

The method 200 can include determining the aggregate count (BLOCK 232). The sequencing system 102 can sum the REF count and the ALT count to determine the aggregate count when the variant types is a structural variant.

The method 200 can include determining gene sequence metrics (BLOCK 234). The gene sequence metrics can include determining an ALT frequency. The sequencing system 102 can determine the ALT frequency as the ALT count divided by the aggregate count for the position. In some implementations, the gene sequence metric can include determining a mean, maximum, minimum, or average coverage depth for the sequence. The sequencing metric can include determining a count of each nucleotide count, and insertion and deletion counts, for every base. Also referring to FIG. 3, the sequencing system 102 can determine the mean, max, or average coverage or read depth for each base pair 302 over each of the sequence listings 300. The sequencing system 102 may only count base pairs 302 that have a quality score above the predetermined threshold. In some implementations, the sequencing system 102 can identify per strand counts to identify strand bias. The sequencing system 102 can also identify clinically relevant variants by identifying alternative calls at the base pair location that occur with a predetermined ALT frequency.

In some implementations, the method 200 can include the sequencing system 102 transmitting the gene sequence metrics to a client device. For example, the sequencing system 102 can transmit the gene sequencing metrics to a laptop or other computing device of the user. In some implementations, the sequencing system 102 can be run as a component of a computing device of the user (e.g., a laptop computer), and the sequencing system 102 can render or display the gene sequence metrics to the user.

FIG. 4 illustrates a block diagram of an example computer system 400. The computer system or computing device 400 can include or be used to implement the system 100 or its components such as the sequencing system 102. For example, the data parser 110, analytics engine 112, reporting engine 104, filtering engine 108 can be components stored on the main memory 415. The computing system 400 includes a bus 405 or other communication component for communicating information and a processor 410 or processing circuit coupled to the bus 405 for processing information. The computing system 400 can also include one or more processors 410 or processing circuits coupled to the bus for processing information. The computing system 400 also includes main memory 415, such as a random access memory (RAM) or other dynamic storage device, coupled to the bus 405 for storing information, and instructions to be executed by the processor 410. The main memory 415 can be or include the data repository 116. The main memory 415 can also be used for storing position information, temporary variables, or other intermediate information during execution of instructions by the processor 410. The computing system 400 may further include a read only memory (ROM) 420 or other static storage device coupled to the bus 405 for storing static information and instructions for the processor 410. A storage device 425, such as a solid state device, magnetic disk or optical disk, can be coupled to the bus 405 to persistently store information and instructions. The storage device 425 can include or be part of the data repository 116.

The computing system 400 may be coupled via the bus 405 to a display 435, such as a liquid crystal display, or active matrix display, for displaying information to a user. An input device 430, such as a keyboard including alphanumeric and other keys, may be coupled to the bus 405 for communicating information and command selections to the processor 410. The input device 430 can include a touch screen display 435. The input device 430 can also include a cursor control, such as a mouse, a trackball, or cursor direction keys, for communicating direction information and command selections to the processor 410 and for controlling cursor movement on the display 435. The display 435 can be part of the sequencing system 102 or other component of FIG. 1, for example.

The processes, systems and methods described herein can be implemented by the computing system 400 in response to the processor 410 executing an arrangement of instructions contained in main memory 415. Such instructions can be read into main memory 415 from another computer-readable medium, such as the storage device 425. Execution of the arrangement of instructions contained in main memory 415 causes the computing system 400 to perform the illustrative processes described herein. One or more processors in a multi-processing arrangement may also be employed to execute the instructions contained in main memory 415. Hard-wired circuitry can be used in place of or in combination with software instructions together with the systems and methods described herein. Systems and methods described herein are not limited to any specific combination of hardware circuitry and software.

Although an example computing system has been described in FIG. 4, the subject matter including the operations described in this specification can be implemented in other types of digital electronic circuitry, or in computer software, firmware, or hardware, including the structures disclosed in this specification and their structural equivalents, or in combinations of one or more of them.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). The operations described in this specification can be implemented as operations performed by a data processing apparatus on data stored on one or more computer-readable storage devices or received from other sources.

The terms “data processing system” “computing device” “component” or “data processing apparatus” encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The components of system 100 can include or share one or more data processing apparatuses, systems, computing devices, or processors.

A computer program (also known as a program, software, software application, app, script, or code) can be written in any form of programming language, including compiled or interpreted languages, declarative or procedural languages, and can be deployed in any form, including as a stand alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs (e.g., components of the sequencing system 102) to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and 13′” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A method to filter sequencing data, comprising:
receiving, by a data processing system, data comprising a plurality of gene sequences, wherein each of the plurality of gene sequences comprise an indication of a chromosome, an indication of a position, a base value, and a quality score;
selecting, by the data processing system, a subset of the plurality of gene sequences, wherein each of the subset of the plurality of gene sequences have the same indication of the chromosome;
filtering, by the data processing system, from the subset of the plurality of gene sequences, gene sequences comprising base values having an associated quality score above a predetermined threshold;
determining, by the data processing system, an aggregate count for each position of the filtered gene sequences;
determining, by the data processing system, an alternative base count for each position of the filtered gene sequences; and
generating, by the data processing system, an identifier of a gene sequence variant, responsive to a ratio of the alternative base count for each position to the aggregate count for each position exceeding a threshold.

2. The method of claim 1, further comprising determining an alternate count for a deletion sequence in the filtered gene sequences.

3. The method of claim 2, wherein the deletion sequence starts at an index neighboring the position.

4. The method of claim 1, further comprising determining an alternate count for an insertion sequence in the filtered gene sequences.

5. The method of claim 4, wherein determining the alternate count for the insertion sequence further comprises identifying an alternate sequence match.

6. The method of claim 1, further comprising identifying a structural variant in the plurality of gene sequences.

7. The method of claim 6, further comprising determining the alternative base count based on the structural variant identified in the plurality of gene sequences.

8. The method of claim 6, wherein determining the aggregate count further comprises counting a match in each of the filtered gene sequences with a CIGAR string.

9. The method of claim 6, wherein determining the aggregate count further comprises counting a deletion, insertion, reference skip, soft clip, or hard clip in each of the subset of the plurality of gene sequences.

10. The method of claim 1, further comprising calculating at least one of a mean read coverage, a max read coverage, or a maximum read coverage for the plurality of gene sequences based on the aggregate count and the alternative base count.

11. The method of claim 1, further comprising calculating a strand bias for the plurality of gene sequences based on the aggregate count and the alternative base count.

12. A system to filter sequencing data, comprising:
a processor in communication with a memory device, the processor executing a data parser and a filtering engine;
wherein the data parser is configured to:
receive, by from the memory device, data comprising a plurality of gene sequences, wherein each of the plurality of gene sequences comprise an indication of a chromosome, an indication of a position, a base value, and a quality score, and
select a subset of the plurality of gene sequences, wherein each of the subset of the plurality of gene sequences have the same indication of the chromosome; and
wherein the filtering engine is configured to:
filter, from the subset of the plurality of gene sequences, gene sequences comprising base values having an associated quality score above a predetermined threshold,
determine an aggregate count for each position of the filtered gene sequences,
determine an alternative base count for each position of the filtered gene sequences, and
generate an identifier of a gene sequence variant, responsive to a ratio of the alternative base count for each position to the aggregate count for each position exceeding a threshold.

13. The system of claim 12, wherein the filtering engine is further configured to determine an alternate count for a deletion sequence in the filtered gene sequences.

14. The system of claim 12, wherein the filtering engine is further configured to determine an alternate count for an insertion sequence in the filtered gene sequences.

15. The system of claim 14, wherein the filtering engine is further configured to determine the alternate count for the insertion sequence by identifying an alternate sequence match.

16. The system of claim 12, wherein the filtering engine is further configured to identify a structural variant in the plurality of gene sequences.

17. The system of claim 16, wherein the filtering engine is further configured to determine the aggregate by counting a match in each of the filtered gene sequences with a CIGAR string.

18. The system of claim 16, wherein the filtering engine is further configured to determine the aggregate count by counting a deletion, insertion, reference skip, soft clip, or hard clip in each of the subset of the plurality of gene sequences.

19. The system of claim 12, wherein the filtering engine is further configured to calculate at least one of a mean read coverage, a max read coverage, or a maximum read coverage for the plurality of gene sequences based on the aggregate count and the alternative base count.

20. The system of claim 12, wherein the filtering engine is further configured to calculate a strand bias for the plurality of gene sequences based on the aggregate count and the alternative base count.


SEQUENCE LISTING (4 sequences)

SEQ ID NO: 1 - DNA, 26 bp, Unknown
  Description of Unknown chromosome sequence
gtcacgtcatccagtcgcaagttagt

SEQ ID NO: 2 - DNA, 26 bp, Unknown
  Description of Unknown chromosome sequence
catccagtcgcaagttagtgtcacgt

SEQ ID NO: 3 - DNA, 24 bp, Unknown
  Description of Unknown chromosome sequence
agtcccaagttagtgtcacgtgtc

SEQ ID NO: 4 - DNA, 24 bp, Unknown
  Description of Unknown chromosome sequence
tcccaagttagtgtcacgtgtctc